(19) United States
(12) Patent Application Publication
(10) Pub. No.: US 2025/0302370 A1
(43) Pub. Date: Oct. 2, 2025

(54) DEVICES, SYSTEMS, AND METHODS FOR CANCER IDENTIFICATION

(71) Applicants: Libby Brooks, Highland, UT (US); Derek Forrester Lewis, Salt Lake City, UT (US); Nathan Andrew Wallace, Salt Lake City, UT (US); Ryleigh Elizabeth Smith, Sandy, UT (US)

(72) Inventors: Libby Brooks, Highland, UT (US); Derek Forrester Lewis, Salt Lake City, UT (US); Nathan Andrew Wallace, Salt Lake City, UT (US); Ryleigh Elizabeth Smith, Sandy, UT (US)

(21) Appl. No.: 19/095,439

(22) Filed: Mar. 31, 2025

Related U.S. Application Data
(60) Provisional application No. 63/572,892, filed on Apr. 1, 2024.

(51) Int. Cl. A61B 5/00; A61B 1/06; A61B 1/303

(52) U.S. Cl. CPC A61B 5/4318; A61B 1/0638; A61B 1/0646; A61B 1/303; A61B 5/0071

(57) ABSTRACT

An imaging device may during a pelvic examination, illuminate a tissue with a blue light, the tissue including at least one of a cervix uteri or vaginal wall. The blue light has a wavelength of between 390 nm and 480 nm and is selected to induce fluorescence in at least one biomarker in cells of the tissue, resulting in fluoresced tissue. An imaging device may capture an image of the tissue including the fluoresced tissue. An imaging device may identify, in the image, an indication of cancer based on the fluoresced tissue.

### CROSS REFERENCE TO RELATED APPLICATIONS

This application claims priority to and the benefit of U.S. Provisional Patent Application No. 63/572,892, titled PORTABLE IMAGER WITH MULTI-COLORED LIGHTS and filed on Apr. 1, 2024, which is hereby incorporated by reference in its entirety.

### BACKGROUND

During the screening and diagnosis process for conditions, such as cervical cancer, it is important to make distinctions between healthy normal tissue, precancerous, and cancerous tissue. In 2022 cervical cancer caused around 350,000 deaths, making it the fourth most deadly cancer in women. Because of this, the World Health Organization labeled cervical cancer as a global health crisis and started an initiative to eliminate this cancer. Screening for cervical cancer is a major component in eliminating cervical cancer because early diagnosis is integral to reducing mortality rates. World-wide, cervical cancer has a 50% survival rate, but if it is diagnosed early patients have a 91% five-year survival rate. To increase early diagnosis, screening rates and accuracy of cervical cancer screening techniques both need to be improved.

Cervical cancer disproportionately affects low-resource areas because of decreased access to screening. Many screening techniques that are common in developed countries require access to histology labs which are often not available. Colposcopy involves viewing the cervix after the application of acetic acid, and visually identifying cancerous or precancerous lesions. However, significant training is required to effectively diagnose cancerous lesions with this technique. Insufficient training leads to both false negatives and false positives. To aid during colposcopy, tools are used to illuminate the cervix uteri and provide magnification. This is accomplished using colposcopes. Colposcopes help to locate potentially problematic tissue and guide placement for samples taken for biopsy.

Existing colposcopes can be mounted on a stand to allow a medical professional to have the free use of their hands or can be handheld. Existing colposcopes can be expensive and not portable. In addition, colposcopy is often an intermediate step in the screening process that leads to biopsies and other laboratory techniques for the further identification of cancerous tissue. This makes cervical cancer screening less accessible to areas in the world that lack resources for laboratory techniques or where medical care facilities are not well established.

### BRIEF SUMMARY

In some aspects, the techniques described herein relate to a method for cervical or vaginal cancer detection. A clinician, during a pelvic examination, illuminates a tissue with a blue light, the tissue including at least one of a cervix uteri or vaginal wall. The blue light has a wavelength of between 390 nm and 480 nm and is selected to induce a fluorescence in at least one biomarker in cells of the tissue, resulting in fluoresced tissue. The clinician captures an image of the tissue including the fluoresced tissue. The clinician identifies, in the image, an indication of cancer based on the fluoresced tissue.

In some aspects, the techniques described herein relate to an imager for identification of cervical or vaginal cancer. The imager includes a light support supporting a plurality of lights. At least one of the plurality of lights includes a blue light having a wavelength of between 390 nm and 480 nm. A camera is arranged to capture an image of a tissue illuminated by at least one of the plurality of lights. A light strip includes a plurality of lenses arranged with the plurality of lights, the plurality of lenses.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF DRAWINGS

In order to describe the manner in which the above-recited and other features of the disclosure can be obtained, a more particular description will be rendered by reference to specific implementations thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example implementations, the implementations will be described and explained with additional specificity and detail through the use of the accompanying drawings in which:

FIG. 1 is a schematic representation of an imaging system, according to at least one embodiment of the present disclosure.

FIG. 2 is a representation of an imaging system, according to at least one embodiment of the present disclosure.

FIG. 3-1 and FIG. 3-2 are representations of imaging systems, according to at least one embodiment of the present disclosure.

FIG. 4 is a flow chart of a method for detecting cancer, according to at least one embodiment of the present disclosure.

FIG. 5 is a representation of a computing system, according to at least one embodiment of the present disclosure.

### DETAILED DESCRIPTION

This disclosure generally relates to devices, systems, and methods for the identification of cancer, including cervical and vaginal cancer. During a pelvic examination of the vaginal walls and uterus, a clinician may illuminate a tissue with blue light. The tissue may include any tissue visible during the pelvic examination, including the cervix uteri and the vaginal walls of the patient. The wavelength of the blue light may be selected to induce fluorescence in certain biomarkers in the tissue. For example, the wavelength of the blue light may be selected to induce fluorescence in at least one of DNA, nicotinamide adenine dinucleotide (NADH or NAD+), nicotinamide adenine dinucleotide phosphate (NADPH or NADP+), flavin adenine dinucleotide (FADH₂ or FADH+), collagen, and so forth. The fluoresced biomarkers may be analyzed identify an indication of the presence and/or extent of cancer. For example, and as discussed in further detail herein, the strength and/or pattern of the fluorescence may be analyzed to determine the presence and/or extent of cancer. This may facilitate an earlier and/or more accurate diagnosis of the presence and/or extent of cancer.

In accordance with at least one embodiment of the present disclosure, an imager may be used to illuminate and capture an image of the tissue. For example, the imager may include one or more lights. The lights may be oriented such that, during a pelvic examination, the lights may illuminate the tissue of a patient's vaginal walls and/or cervix uteri. The imager may include a camera or other image capturing device, which may collect an image of the tissue. In some embodiments, a clinician may view the image of the tissue on a display of the imager. In some embodiments, the imager may store the image and/or transmit the image to a remote computing device, such as a laptop computer, a desktop computer, a cloud computing system, a mobile device, a tablet, any other computing device, and combinations thereof. This may facilitate the review and analysis of the illuminated tissue by others and at a time after the examination, including by specialists and/or third parties. In some embodiments, illuminating the tissue with blue light may facilitate more accurate and/or more reliable identification of cancerous or pre-cancerous tissue, reducing or preventing false positives and/or false negatives. Put another way, illuminating the tissue may facilitate distinguishing between pre-cancerous, cancerous, and normal tissue.

In some embodiments, the techniques of the present disclosure may be implemented in disadvantaged areas. For example, areas with limited access to medical care, or that do not have funding or availability for extensive tissue sampling and testing, may utilize the imaging devices and methods described herein to more reliably identify cancer at the cervix uteri and/or the vaginal walls. This may allow clinicians to intervene earlier (or at all) for patients that otherwise may not have identified cancerous or pre-cancerous tissue in sufficient time to treat the tissue.

In some embodiments, the imager and/or other image processing system may further process the image of the fluorescing tissue. For example, the image processing system may apply one or more filters, diffusers, brightness adjustments, color adjustments, polarizers, or other image adjusters. This may serve to emphasize, highlight, contrast, or otherwise facilitate the identification of cancer in the tissue.

In some embodiments, after identifying the indication of cancer, the clinician may provide and/or implement a recommendation for additional testing and/or therapeutic care. For example, the clinician may collect a tissue sample based on the indication of cancer. In some examples, the clinician may prescribe and/or administer certain therapeutic agents (e.g., medications) based on the indication of cancer. In some examples, the clinician may perform and/or recommend a procedure based on the indication of cancer. In this manner, the clinician may provide care for the patient based on the early diagnosis of cancer.

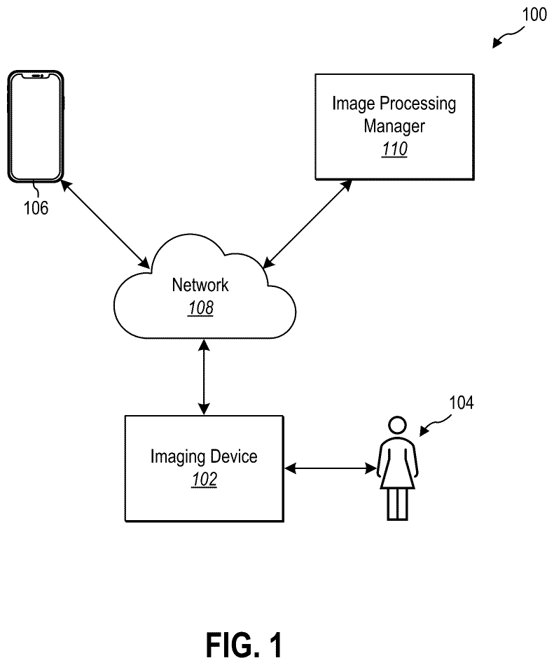

FIG. 1 is a schematic representation of an imaging system 100, according to at least one embodiment of the present disclosure. The imaging system 100 includes an imaging device 102. The imaging device 102 may be an imaging device configured to capture images of a patient 104 during a pelvic examination. For example, the imaging device 102 may be used to capture images of a cervix uteri of the patient 104. In some examples, the imaging device 102 may be used to capture images of a vaginal wall of the patient 104. The imaging device 102 may include any type of imaging device. For example, the imaging device 102 may include a colposcope. In some examples, the imaging device 102 may include a handheld colposcope. In some examples, the imaging device 102 may include a desk mounted colposcope, including imaging equipment located on a platform or a desk and connected to a scope used during examination of the patient 104. In some embodiments, the imaging device 102 may be used in conjunction with a speculum. In some embodiments, the imaging device 102 may image the cervix uteri and/or the vaginal wall without a speculum.

In some embodiments, a clinician may prepare the tissue of the patient 104 prior to imaging. For example, the clinician may wash the tissue with a preparation solution. Such a preparation solution may improve the image capture, such as by dehydrating the cells. The preparation solution may include one or more of acetic acid (vinegar), iodine, any other preparation solution, and combinations thereof, including mixtures and/or sequential washings of any of the foregoing.

During capture of the image, the imaging device 102 may illuminate tissue of the patient 104 with one or more blue lights. The blue lights may cause or induce fluorescence in certain tissues of the patient 104. For example, the blue lights may cause fluorescence in biomarkers of the cells of the patient. In some embodiments, the blue lights may facilitate illumination of biomarkers below the surface of the tissue. For example, the blue lights may penetrate 1 microns, 2 microns, 3, microns, 4 microns, or 5 microns below the surface of the tissue. This may help to increase the identification of the target biomarkers that are indicative of cancerous or pre-cancerous tissue.

The imaging device 102 may facilitate the remote viewing of the fluoresced tissue. For example, the imaging device 102 may include a display, and the clinician may view the fluoresced tissue in the display. In some embodiments, the imaging device 102 may capture an image of the tissue. For example, the imaging device 102 may include a camera or other image capture device that may capture and store and/or transmit the captured image for later analysis. For example, the imaging device 102 may include internal storage. When the image capture device on the imaging device 102 captures the image, the imaging device 102 may store the image in the internal storage. In some examples, the imaging device 102 may include a communication system, including a wired and/or wireless (e.g., Wi-Fi, Bluetooth, cellular, or other wireless communication system) communication system to transmit the captured and/or stored image to a user device 106 and/or cloud storage. In some embodiments, the imaging device 102 may transmit the captured and/or stored image to the user device 106 over a network 108, such as the Internet.

A clinician, such as a medical technician, a nurse, a nurse practitioner, a doctor, a specialist, or other clinician, may examine the fluoresced tissue from the image (e.g., at the imaging device 102, at the user device 106, or at another imaging system configured to display the image for review) to identify the presence of cancer or pre-cancerous tissue. In accordance with at least one embodiment of the present disclosure, the blue light may cause or induce a greater amount of fluorescence in cancerous or pre-cancerous tissue. For example, cancerous or pre-cancerous tissue may include a higher concentration of DNA or other biomarkers, as discussed herein. The clinician may identify these higher concentrations based on the increased intensity of fluoresced tissue. For example, the clinician may identify the higher concentrations of the biomarker using an increased brightness of the fluoresced tissue, a higher contrast between the fluoresced tissue and the surrounding tissue, a color of the fluoresced tissue, a fluorescence wavelength of the fluorescence from the fluoresced tissue, a pattern of the fluoresced tissue, a total amount of the fluoresced tissue, any other pattern of the fluoresced tissue, and combinations thereof, including inclusions and exclusions of any of the foregoing.

In accordance with at least one embodiment of the present disclosure, the imaging system 100 may further include an image processing manager 110. The image processing manager 110 may process the captured image of the tissue. For example, the image processing manager 110 may perform one or more operation on the image, including apply a filter, apply a diffuser, change the contrast, change the brightness, perform any other operation on the image, and combinations thereof. This may facilitate emphasizing, highlighting, or otherwise improving the identification of cancerous or pre-cancerous tissue using the image collected by the imaging device 102.

In accordance with at least one embodiment of the present disclosure, the image captured by the imaging device 102 may facilitate an identification of the location of the cancerous or pre-cancerous tissue. For example, the fluoresced tissue may highlight or identify the locations of the tissue that are cancerous or pre-cancerous based on the areas that have the highest intensity of fluoresced tissue. Identifying the location of the cancerous or pre-cancerous tissue may enable a clinician to more accurately collect tissue samples, perform biopsies, remove the cancerous or pre-cancerous tissue, and so forth. In some embodiments, a clinician may perform a biopsy or other procedure while illuminating the tissue with blue light.

In accordance with at least one embodiment of the present disclosure, the clinician may track the progression of a cancer or other disease using images captured by the imaging device 102. For example, the clinician may capture images at subsequent visits to the clinician, which may be separated by a period of time. The clinician may compare fluoresced tissue in subsequent images and identify the introduction of and/or change in intensity of fluoresced tissue. In this manner, the clinician may identify the progression of diseased tissue. In some embodiments, the clinician may monitor pre-cancerous tissue or tissue that is not pre-cancerous, but worthwhile to watch. This may facilitate targeted care. In some embodiments, this may reduce or prevent unnecessary procedures on the imaging device 102.

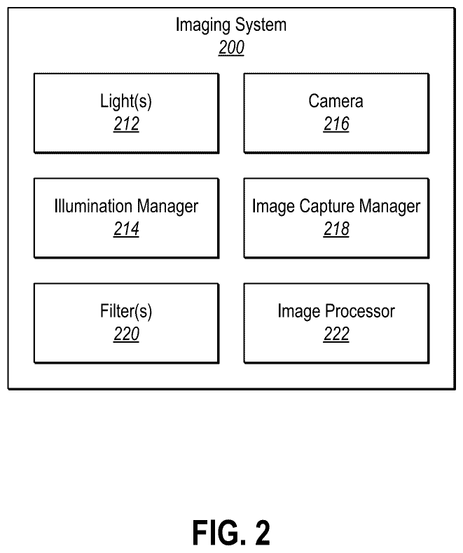

FIG. 2 is a representation of an imaging system 200, according to at least one embodiment of the present disclosure. Each of the components of the imaging system 200 can include software, hardware, or both. For example, the components can include one or more instructions stored on a computer-readable storage medium and executable by processors of one or more computing devices, such as a client device or server device. When executed by the one or more processors, the computer-executable instructions of the imaging system 200 can cause the computing device(s) to perform the methods described herein. Alternatively, the components can include hardware, such as a special-purpose processing device to perform a certain function or group of functions. Alternatively, the components of the imaging system 200 can include a combination of computer-executable instructions and hardware.

Furthermore, the components of the imaging system 200 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

As discussed herein, the imaging system 200 may include one or more lights 212 to illuminate a tissue. The lights 212 may include any type of light. For example, the lights 212 may include one or more light emitting diodes (LEDs), organic light emitting diodes (OLED), or other lights. The lights 212 may be selected to emit light in a specific wavelength or range of wavelengths. For example, the lights 212 may be blue lights, and may be configured or arranged to emit light in the blue portion of the visible light spectrum. In some embodiments, the wavelength may be in a range having an upper value, a lower value, or upper and lower values including any of 390 nm, 400 nm, 410 nm, 420 nm, 430 nm, 440 nm, 450 nm, 460 nm, 470 nm, 480 nm, or any value therebetween. For example, the wavelength may be greater than 390 nm. In another example, the wavelength may be less than 480 nm. In yet other examples, the wavelength may be any value in a range between 390 nm and 480 nm. In some embodiments, it may be critical that the wavelength is between 400 nm and 450 nm to target the biomarkers that are indicative of cancer in the cervix uteri and/or the vaginal wall. In some embodiments, the wavelength may be 430 nm. In some embodiments, the wavelength may be 447 nm.

Certain molecules fluoresce when electrons in the highest occupied molecular orbital are hit with/excited by a photon of light. These electrons then jump up in energy and configuration to the lowest unoccupied molecular orbital. When those electrons relax back down into the highest occupied molecular orbital they release a photon of a longer wavelength than that of the photon they were hit with. The excitation and subsequent emission of photons with different wavelengths is fluorescence. The difference between the energy of the highest occupied molecular orbital and that of the lowest unoccupied molecular orbital is what determines the wavelength of light needed to excited the electrons and also determines the wavelength of light emitted. The molecular orbitals are unique to each molecule and are highly impacted by conjugated pi systems, aromatic rings, and the presence of atoms other than carbon and hydrogen.

In accordance with at least one embodiment of the present disclosure, the wavelength of one or more of the lights 212 may be selected based on a targeted biomarker. For example, the wavelength of the lights 212 may be selected to induce the highest fluorescence response in the targeted biomarker. As a specific, non-limiting example, FAD may fluoresce (e.g., emit light based on an illumination of blue light) at 505 nm, NADH may fluoresce at 460 nm, and collagen may fluoresce at 400 nm.

Conventional tissue imaging techniques may utilize high-energy light. For example, conventional tissue imaging techniques may identify vascular structure or other structures in the cervix uteri and/or the vaginal wall using ultraviolet light (e.g., light with a wavelength smaller than 390 nm). The ultraviolet light may irritate or damage the sensitive tissue of the vaginal wall and/or cervix uteri. Utilizing lights 212 having a wavelength in the blue spectrum may enable the clinician to identify the presence of cancerous and/or pre-cancerous while reducing or preventing damage to the patient from the emitted light.

In accordance with at least one embodiment of the present disclosure, the lights 212 may include multiple wavelengths of lights. For example, the lights 212 may include multiple lights on an imaging device. Different lights may have different wavelengths. For example, a first light may have a first wavelength, a second light may have a second wavelength, and so forth. The different wavelengths may be targeted at different biomarkers and/or configured to both complementarily highlight a single target biomarker. In some embodiments, multiple lights may have the same wavelength. In some embodiments, one or more of the lights may include white light, or multi-spectrum light that covers all or most of the visible spectrum.

The imaging system 200 may further include an illumination manager 214. The illumination manager 214 may control the actuation (e.g., turning on) and deactuation (e.g., turning off) of the various lights 212. In some embodiments, the illumination manager 214 may individually actuate each light. In some embodiments, the illumination manager 214 may simultaneously actuate multiple lights. In some embodiments, the illumination manager 214 may actuate or deactuate any combinations of lights 212 based on the desires of the clinician and the specific combination of wavelengths (including white light) desired for a particular image, review, or other process. For example, a clinician may begin a pelvic examination using white light to move the speculum and/or imaging device into position. The white light may produce a glare on the patient's tissue. The clinician may switch between lights 212 to reduce glare, generate images, and/or position the instruments for the pelvic examination.

The imaging system 200 may further include a camera 216 or other image collection system. The camera 216 may capture an image of the tissue. to capture an image, the illumination manager 214 may cause one or more of the lights 212 to illuminate the tissue. An image capture manager 218 may instruct the camera 216 to capture the image. The camera 216 may include any image collection system that may record reflected light. In some embodiments, the camera 216 may be specially designed or configured to capture or collect fluoresced light. For example, the camera 216 may include fluorescent detectors in combination with band-pass filters, such as photo multiplying tubes, silicon-based solid-state detectors, or other fluorescent detectors that may enhance the detection and visual representation of detected fluorescent response of biochemical features.

In some embodiments, the image capture manager 218 may cause one or more filters 220 to overlay the camera 216 and/or the lights 212. The filters 220 may filter the light emitted by the lights 212 and/or the fluoresced light received by the camera 216. This may enable the clinician to isolate, emphasize, or highlight certain aspects of the fluoresced tissue, thereby increasing the accuracy and/or reliability of the identification of cancer or pre-cancer in the tissue.

In accordance with at least one embodiment of the present disclosure, an image processor 222 may at least partially process the image captured by the camera 216. For example, the image processor 222 may apply, after the image is captured, one or more of the filters 220 to the image, apply a diffuser to the image, adjust the contrast, brightness, color, or other aspect of the captured image. Processing the image may help to isolate, highlight, emphasize, or otherwise improve identification of the cancerous and/or pre-cancerous tissue in the patient.

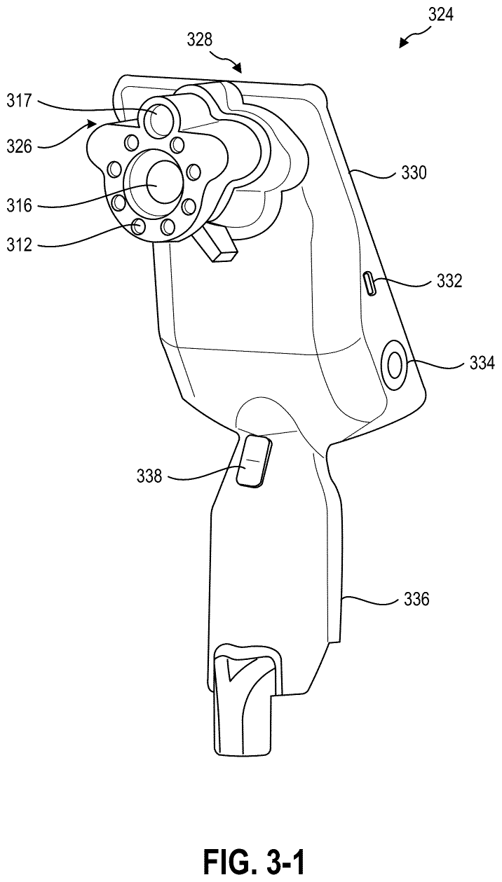

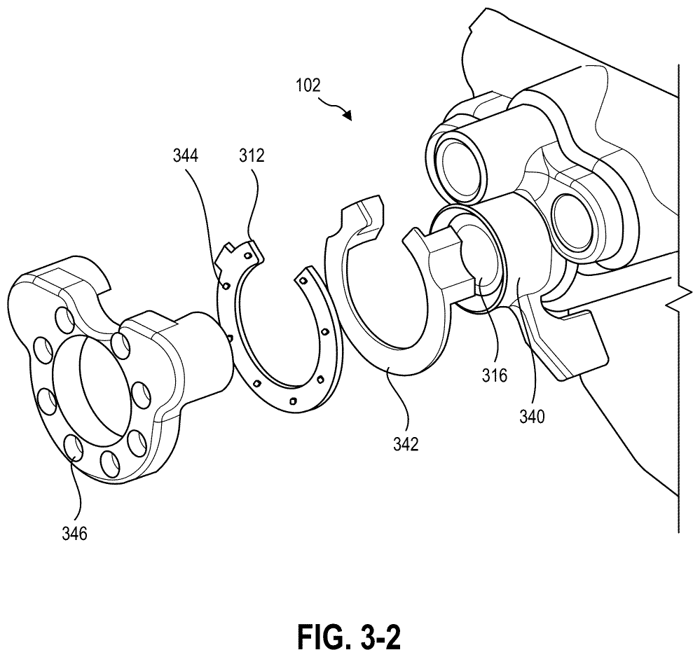

FIG. 3-1 is a perspective view of an imaging device 324, according to at least one embodiment of the present disclosure. The imaging device 324 includes an image capture system 326. The image capture system 326 includes a camera 316. The camera 316 may facilitate the capture of one or more images of a patient's cervix uteri and/or vaginal walls during a pelvic examination. The image capture system 326 further includes one or more lights 312. The lights 312 may be located proximate the camera 316 to illuminate the tissue to be captured by the camera 316. In some embodiments, the imaging device 324 may further include one or more fluorescent detectors 317. The fluorescent detectors 317 may include components specially designed and selected to capture emitted fluorescent light, such as photo multiplying tubes or silicon-based solid-state detectors.

As discussed herein, the lights 312 may include blue lights. Blue lights may cause certain biomarkers in the tissue of the patient to fluoresce. The camera 316 may capture an image of the tissue while the lights 312 are illuminating the tissue. In some embodiments, the camera 316 may capture an image of the tissue while the lights 312 are causing the tissue to fluoresce based on one or more blue lights illuminating the tissue. As discussed herein, the imaging device 324 may include multiple lights 312. For example, in the imaging device 324 illustrated, the image capture system 326 includes 8 lights. However, it should be understood that any number of lights 312 may be used, including 1, 2, 3, 4, 5, 6, 7, 8, 9, 10, 15, 20, or more lights 312.

As discussed herein, the lights 312 may include more than one wavelength of light. As a specific, non-limiting example, the lights 312 of the imaging device 324 illustrated include four blue lights and four white lights, spaced alternately around the camera 316. However, it should be understood that any combination of colors of lights may be used arranged in any spacing. For example, the image capture system 326 may include lights 312 in different wavelengths in the blue spectrum, the image capture system 326 may include lights 312 in different color spectrums. In some examples, the image capture system 326 may include any pattern of lights 312, including blue lights arranged on one half and white lights on the other half, or other arrangement of lights 312.

In some embodiments, the image capture system 326 may include one or more optical elements, such as lenses, grisms, prisms, diffractors, diffusers, or other optical elements to direct light emitted by the lights 312 to illuminate the tissue. For example, the optical elements may facilitate a targeted illumination of specific portions of the tissue, the optical elements may facilitate a broad, even illumination of the entire visible area of the tissue, balanced illumination of the tissue, and so forth. In some embodiments, each of the lights 312 may have the same optical element. In some embodiments, different lights 312 may have different optical elements. The lights 312 may be independently actuatable, such that a particular light 312 associated with a particular optical element may be illuminated independently of the remaining lights 312.

In accordance with at least one embodiment of the present disclosure, the imaging device 324 may include a display 328. The display 328 may facilitate viewing of the tissue. For example, the display 328 may display a view as seen by the camera 316. In this manner, during a pelvic examination, the clinician may view the area to be imaged. In some embodiments, the display 328 may further display the captured images of the tissue. For example, the operator may view the captured images on the display 328 to determine if the captured image is sufficient for the identification of cancerous and/or pre-cancerous tissue.

The imaging device 324 may further include a user interface 330. The user interface 330 may allow the clinician to control operation of the imaging device 324. For example, the user interface 330 may control illumination of the various lights 312, capturing of images using the camera 316, reviewing previously captured images, transmitting images to a remote computing device, and so forth. Images may further be captured using a capture button 338.

The imaging device 324 may include one or more data transfer ports 332, such as a USB port, coaxial port, or other data transfer port. Power or other operation of the imaging device 324 may be controlled via a power button 334. During operation, the clinician may grip the imaging device 324 at a handle 336. The imaging device 324 may be powered using a wired power connection and/or an internal battery, such as a rechargeable battery.

FIG. 3-2 is an exploded view of the image capture system 326 of FIG. 301. The image capture system 326 includes a device attachment 340. The device attachment 340 may be arranged and configured to secure the lights to the imaging device 324. The device attachment 340 may be centered around the camera 316 to facilitate illumination of the tissue in the field of view of the 316. A light support structure 342 may support a light strip 344 and connect or secure the light strip 344 to the device attachment 340. The light strip 344 may include the lights 312. In some embodiments, the light strip 344 may include a printed circuit board (PCB) that may include electronics used to selectively turn on and off the lights 312. An optical element support 346 may support one or more optical elements that cover the lights 312. For example, as discussed herein, the optical element support 346 may include one or more lenses, grisms, prisms, diffractors, diffusers, or other optical elements to direct light emitted by the lights 312 to illuminate the tissue. The optical element support 346 may facilitate replacement and/or customization of the optical elements based on the preferences of the clinician.

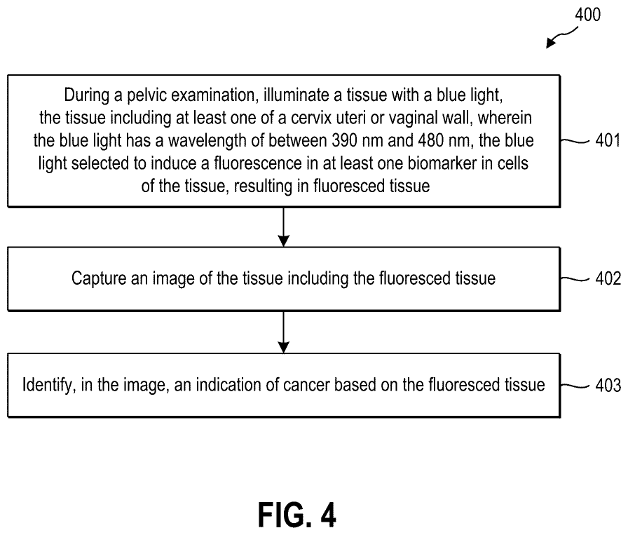

FIG. 4, the corresponding text, and the examples provide a number of different methods, systems, devices, and computer-readable media of the imaging system. In addition to the foregoing, one or more embodiments can also be described in terms of flowcharts comprising acts for accomplishing a particular result, as shown in FIG. 4. FIG. 4 may be performed with more or fewer acts. Further, the acts may be performed in differing orders. Additionally, the acts described herein may be repeated or performed in parallel with one another or parallel with different instances of the same or similar acts.

As mentioned, FIG. 4 illustrates a flowchart of a series of acts or a method 400 for cervical or vaginal cancer detection, according to at least one embodiment of the present disclosure. While FIG. 4 illustrates acts according to one embodiment, alternative embodiments may omit, add to, reorder, and/or modify any of the acts shown in FIG. 4. The acts of FIG. 4 can be performed as part of a method. Alternatively, a computer-readable medium can comprise instructions that, when executed by one or more processors, cause a computing device to perform the acts of FIG. 4. In some embodiments, a system can perform the acts of FIG. 4.

A clinician may, during a pelvic examination, illuminate a tissue with a blue light at 401. The tissue includes at least one of a cervix uteri or a vaginal wall. The blue light has a wavelength of between 390 nm and 480 nm and is selected to induce a fluorescence in at least one biomarker in cells of the tissue, resulting in fluoresced tissue. The clinician may capture an image of the tissue including the fluoresced tissue at 402. The clinician may identify, in the image, an indication of cancer based on the fluoresced tissue at 403.

In some embodiments, the biomarker includes DNA, NAD, FAD, collagen, or combinations thereof. In some embodiments, identifying the indication of cancer includes identifying the indication of cancer based on a total amount of fluoresced tissue. In some embodiments, the indication of cancer is identified based on a brightness of the fluoresced tissue, a location of the fluoresced tissue, a wavelength of the fluoresced tissue, and so forth. In some embodiments, after capturing the image of the tissue, the clinician may process the image of the tissue. In some embodiments, the clinician may prepare the tissue. In some embodiments, the clinician may distinguish between pre-cancerous, cancerous, and normal tissue.

In some embodiments, the clinician may capture a second image of the tissue with a second light illuminating the tissue. The second light may be a blue light, and the second image, in combination with the first image, may be used to identify the indication of cancer.

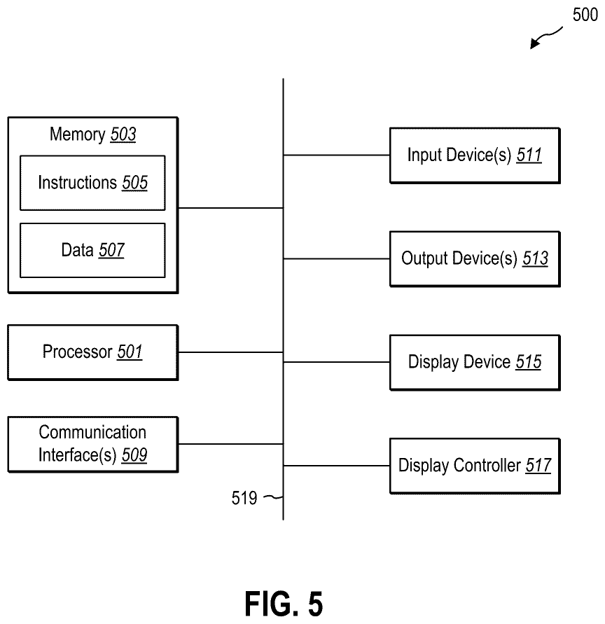

FIG. 5 illustrates certain components that may be included within a computer system 500. One or more computer systems 500 may be used to implement the various devices, components, and systems described herein.

The computer system 500 includes a processor 501. The processor 501 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 501 may be referred to as a central processing unit (CPU). Although just a single processor 501 is shown in the computer system 500 of FIG. 5, in an alternative configuration, a combination of processors (e.g., an ARM and DSP) could be used.

The computer system 500 also includes memory 503 in electronic communication with the processor 501. The memory 503 may be any electronic component capable of storing electronic information. For example, the memory 503 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 505 and data 507 may be stored in the memory 503. The instructions 505 may be executable by the processor 501 to implement some or all of the functionality disclosed herein. Executing the instructions 505 may involve the use of the data 507 that is stored in the memory 503. Any of the various examples of modules and components described herein may be implemented, partially or wholly, as instructions 505 stored in memory 503 and executed by the processor 501. Any of the various examples of data described herein may be among the data 507 that is stored in memory 503 and used during execution of the instructions 505 by the processor 501.

A computer system 500 may also include one or more communication interfaces 509 for communicating with other electronic devices. The communication interface(s) 509 may be based on wired communication technology, wireless communication technology, or both. Some examples of communication interfaces 509 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 500 may also include one or more input devices 511 and one or more output devices 513. Some examples of input devices 511 include a keyboard, mouse, microphone, remote control device, button, joystick, trackball, touchpad, and lightpen. Some examples of output devices 513 include a speaker and a printer. One specific type of output device that is typically included in a computer system 500 is a display device 515. Display devices 515 used with embodiments disclosed herein may utilize any suitable image projection technology, such as liquid crystal display (LCD), light-emitting diode (LED), gas plasma, electroluminescence, or the like. A display controller 517 may also be provided, for converting data 507 stored in the memory 503 into text, graphics, and/or moving images (as appropriate) shown on the display device 515.

The various components of the computer system 500 may be coupled together by one or more buses, which may include a power bus, a control signal bus, a status signal bus, a data bus, etc. For the sake of clarity, the various buses are illustrated in FIG. 5 as a bus system 519.

Embodiments of the present disclosure may thus utilize a special purpose or general-purpose computing system including computer hardware, such as, for example, one or more processors and system memory. Embodiments within the scope of the present disclosure also include physical and other computer-readable media for carrying or storing computer-executable instructions and/or data structures, including applications, tables, data, libraries, or other modules used to execute particular functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily store or carry, software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A “network” or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for cervical or vaginal cancer detection, the method comprising:
during a pelvic examination, illuminating a tissue with a blue light, the tissue including at least one of a cervix uteri or vaginal wall, wherein the blue light has a wavelength of between 390 nm and 480 nm, the blue light selected to induce a fluorescence in at least one biomarker in cells of the tissue, resulting in fluoresced tissue;
identifying, in the image, an indication of cancer based on the fluoresced tissue.

2. The method of claim 1, wherein the at least one biomarker includes DNA.

3. The method of claim 1, wherein the at least one biomarker includes at least one of NAD, FAD, or collagen.

4. The method of claim 1, wherein the blue light has the wavelength of 430 nm.

5. The method of claim 1, wherein identifying the indication of cancer includes identifying the indication of cancer based on a total amount of the fluoresced tissue.

6. The method of claim 1, wherein identifying the indication of cancer includes identifying the indication of cancer based on a brightness of the fluoresced tissue.

7. The method of claim 1, wherein identifying the indication of cancer includes identifying the indication of cancer based on a location of the fluoresced tissue.

8. The method of claim 1, wherein identifying the indication of cancer includes identifying the indication of cancer based on a fluorescence wavelength of the fluoresced tissue.

9. The method of claim 1, further comprising, after capturing the image of the tissue, processing the image of the tissue, wherein processing the image of the tissue includes at least one of filtering, adjusting a brightness, polarizing, or adjusting a contrast of the image.

10. The method of claim 1, further comprising preparing the tissue, including applying acetic acid to the tissue.

11. The method of claim 1, wherein identifying the indication of cancer includes highlighting features of the tissue that allow a clinician to distinguish between pre-cancerous, cancerous, and normal tissue.

12. The method of claim 1, further comprising, using the indication of cancer, administering a therapeutic agent to a patient.

13. The method of claim 1, wherein the blue light includes a first blue light having a first wavelength and the image includes a first image, and further comprising:
illuminating the tissue with a second wavelength of light; and
capturing a second image illuminated by the second wavelength of light, wherein identifying the indication of cancer includes identifying the indication of cancer using the first image and the second image.

14. The method of claim 13, wherein the second wavelength of light is between 390 nm and 480 nm.

15. The method of claim 1, further comprising capturing an image of the tissue including the fluoresced tissue.

16. The method of claim 15, wherein capturing the image includes capturing the image using one or more fluorescent detectors.

17. The method of claim 15, further comprising displaying the image on a display of an imaging device.

18. An imager for identification of cervical or vaginal cancer, the imager comprising:
a light support supporting a plurality of lights, at least one of the plurality of lights including a blue light having a wavelength of between 390 nm and 480 nm;
a camera arranged to capture an image of a tissue illuminated by at least one of the plurality of lights; and
a light strip including a plurality of lenses arranged with the plurality of lights, the plurality of lenses.

19. The imager of claim 18, wherein the blue light includes a first blue light and the wavelength includes a first wavelength, and further comprising a second blue light having a second wavelength.

20. The imager of claim 18, further comprising an optical element support including at least one filter between the plurality of lights and the tissue.
